(19) United States
(12) Patent Application Publication
(10) Pub. No.: US 2002/0035351 A1
(43) Pub. Date: Mar. 21, 2002

(54) Single barrel double chamber syringe

(76) Inventor: Christopher Charles Lodice, Picayune, MS (US)

Correspondence Address: Christopher Lodice, 274 Magnolia Drive, Picayune, MS, 39466, US

(21) Appl. No.: 09/953,149

(22) Filed: Sep. 17, 2001

Related U.S. Application Data
(60) Provisional application No. 60/233,224, filed on Sep. 16, 2000.

(51) Int. Cl. A61M 5/315

(52) U.S. Cl. 604/221

(57) ABSTRACT

The existing syringes in use today consist of a single barrel with only one chamber and one plunger. The “Single Barrel Double Chamber Syringe” is a modified version of the existing syringe in that it uses a single barrel syringe separated into two chambers by the use of a hollow cone shaped floating plunger. The hollow floating plunger creates two independent chambers capable of delivering two medications in one motion without mixing of the fluids. The hub of the syringe has also been modified with a needle that has a sharp point facing to the inside of the syringe and has a small hole bored into the side near the base. This will allow the hollow floating plunger to be pierced once the first chamber is emptied. The hole in the base of the needle will relieve the pressure in the first chamber after the needle has passed through the hollow floating plunger while allowing any remaining fluid to be expelled. Once the first chamber has been emptied, the second chamber will begin to empty just as the first, until the manual plunger reaches the hollow floating plunger. The manual plunger will also be cone shaped so it will fit snugly into the hollow side of the floating plunger. This will allow as much fluid as possible in chamber two to be expelled.

### BRIEF DESCRIPTION OF THE DRAWINGS

These and other features of the present invention will be more clearly understood from a consideration of the following description, taken in connection with the accompanying drawing in which:

FIGS. 1a-1 c illustrate a single barrel double chamber syringe according to a preferred embodiment of the invention and helpful in an understanding of its operation; and

FIGS. 2a-2c are views of several of the component parts of the syringe of FIGS. 1a-1c, partially in section form, helpful in an understanding of the two chambered segregated fluid delivery system.

### DETAILED DESCRIPTION OF INVENTION

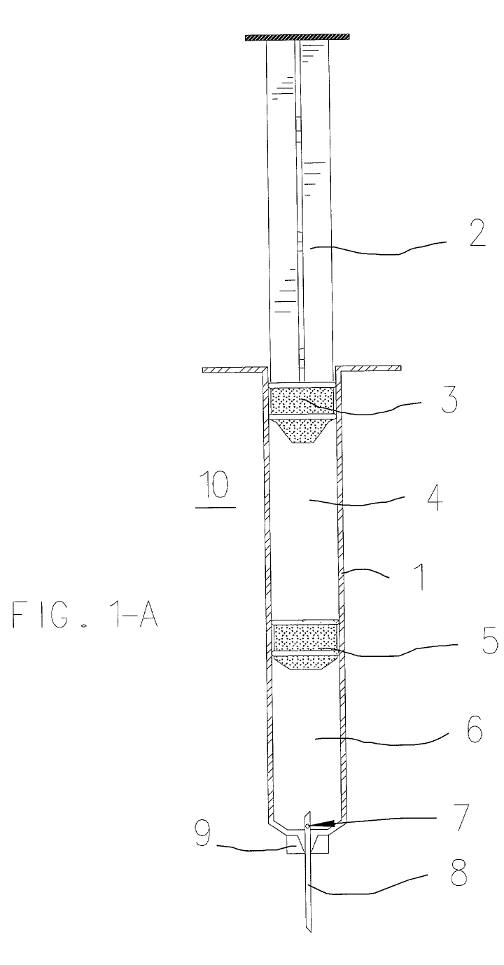

Referring to FIGS. 1a-1c, the single barrel double chamber syringe of the invention is illustrated by the reference numeral 10, having a barrel 1 and plunger 2 by means of which any fluids within the barrel 1 are forced outwardly through its hollow needle 8. To effectuate this, a push stopper 3 is shown coupled to the plunger 2. In accordance with the invention, the push stopper 3 is preferably made of a pliable material to maintain a fluid tight seal around the outer periphery.

Also shown in FIGS. 1a-1c is a hollow floating plunger 5 within the barrel 1, likewise composed of a pliable material to maintain a fluid tight seal around the outer periphery, and forming a first compartment 4 between the stoppers 3, 5 within which any included fluid or solution becomes separated.

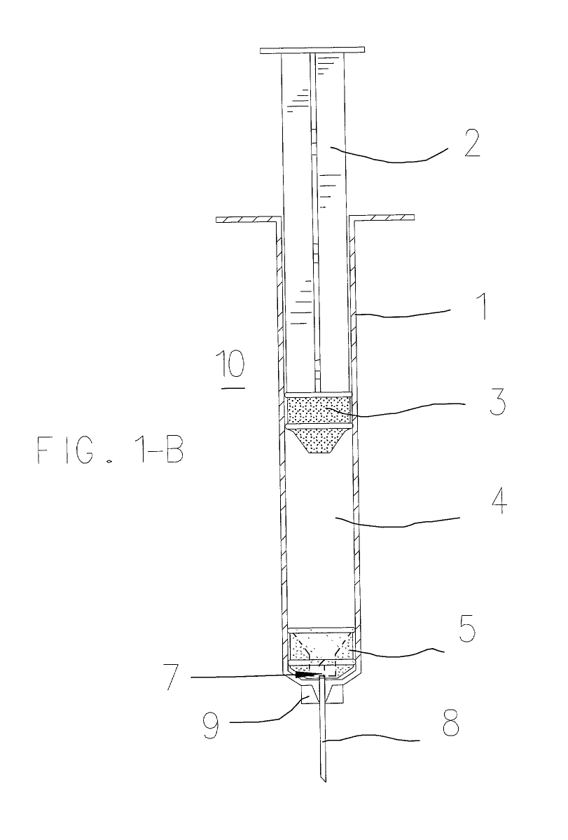

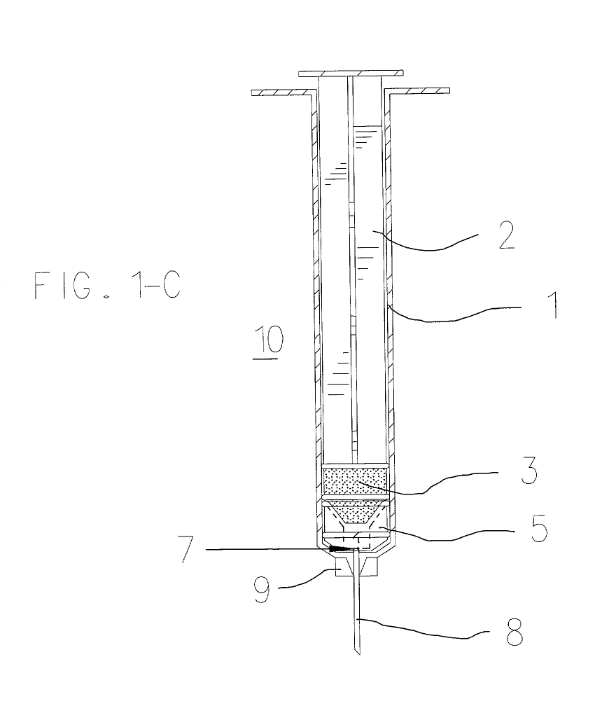

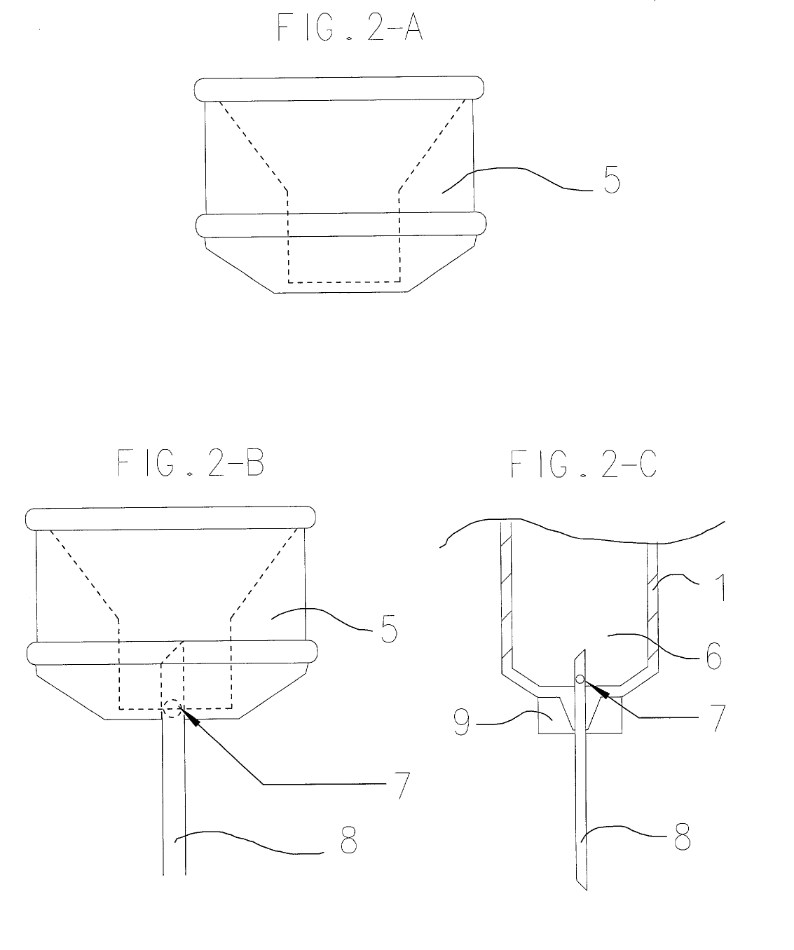

Additionally, in accordance with the invention, the hollow floating plunger will be illustrated in FIGS. 2a-2b, as will be appreciated, a piercing device 8 is inserted within, or otherwise affixed to, the hub 9 of the syringe 1. In this manner, it will be seen that compartment 6 is formed, of which may be pre-filled or loaded with a fluid, or medication of choice.

Thus, and in order to carry out the objectives of the invention, certain other requirements will be seen to be necessary. First of all, the length of the hollow needle 8 is selected of sufficient dimension to pierce the hollow floating plunger 5, but of such limited length to stop short of the push stopper 3. The hollow needle 8 should have a relief port hole 7 bored into the side and located close to the hub 9 of syringe 1 in chamber 6 as illustrated in FIG. 2c.

As thus described, the arrangement of FIGS. 1a-1c will be seen to represent a syringe having two segregated compartments 4 and 6, and with a delivery needle 8 that has a piercing tip and a relief port hole 7 of a type typically found in a prior-art cartridge syringe frequently used. A cap (not shown) would normally be placed over the delivery tip (needle or needless system), so as to create a fluid tight compartment when not in use, but one where the cap could be removed for appropriate delivery of the fluid to the person or body where the fluid injection is to occur.

With such cap then in place over the delivery tip each of the compartments 4 and 6 will be seen to be fluid tight—and, for the injection of medication in chamber 6, and a normal saline flush injection in compartment 4 with each compartment being segregated one from another. But, by the removing the cap and applying pressure on the plunger 2, the hollow floating plunger 5 will be seen to advance as the fluid in chamber 6 closest to the delivery tip is allowed to exit. With constant pressure on the plunger, then all the fluid in compartment 6 will first evacuate under the force applied by the hollow floating plunger 5. As the plunger 2 continues to force the hollow floating plunger 5 to the hub 9 of the syringe 1 the hollow needle 8 punctures the thin membrane of the hollow floating plunger 5. The relief porthole 7 allows the excess pressurized fluid to be expelled to complete the motion of the hollow floating plunger 5 as it becomes flush with the hub 9 of syringe 1.

As the plunger action continues, the segregated fluid in chamber 4 is expelled through the hollow needle 8 until the push stopper 3 is flushed against the hollow floating plunger 5. Thus, by a single, continuous actuation of the plunger 2, a sequential delivery of the fluids and or medications in compartments 4 and 6 are expelled, without the need for any refilling or reloading of the syringe in use, and without any need for disconnecting it from the injection site.

What I claim as my invention is:

1. A syringe, used by paramedics, nurses, and doctors in the medical field that will allow a medication to be administered with a saline bolas to flush the medication in one easy push without the risk of time delay and awkwardness of having to use two syringes that need to be prepared and administered separately.
